(19) United States
(12) Patent Application Publication
(10) Pub. No.: US 2023/0355150 A1
(43) Pub. Date: Nov. 9, 2023

(54) METHOD AND DEVICE FOR DETERMINING A MENTAL STATE OF A USER

(71) Applicant: Sara VIELE, London (GB)

(72) Inventor: Sara VIELE, London (GB)

(21) Appl. No.: 18/022,544

(22) PCT Filed: Jul. 9, 2021
(86) PCT No.: PCT/EP2021/069159

§ 371 Date: Feb. 22, 2023

(30) Foreign Application Priority Data
Aug. 24, 2020 (GB) 2013220.5

(51) Int. Cl. A61B 5/0533; A61B 5/024; A61B 5/00; A61B 5/16; A61B 5/01

(52) U.S. Cl. CPC A61B 5/165; A61B 5/0024; A61B 5/01; A61B 5/024; A61B 5/0533; G06F 2203/011

(57) ABSTRACT

The present invention relates to devices and methods for determining an emotional state of a user. Specifically, a computer-implemented method is provided comprising measuring a galvanic skin response voltage and a skin temperature of the user by first and second sensors; passing these values to a processor for comparison to a plurality of predefined threshold values, each being associated with one or more likely emotional states;; and based on the comparisons of the first and second measured values, determining an emotional state of the user, and in response to the determination that the user is in a particular emotional state, initiating one or more protocols for adapting the environment of the user to that emotional state. A device configured to carry out the operations required to perform the method is also provided.

### CROSS REFERENCES TO RELATED APPLICATIONS

The present application claims the benefit and priority of UK application no. GB2013220.5, filed 24 Aug. 2020.

### FIELD OF INVENTION

The present invention relates generally to methods and devices for detecting a user’s mood. More specifically, the present invention relates to the application of multiple sensors to a user’s skin and comparing the measured values to thresholds known to be associated with particular emotions to determine how the user is feeling before they are aware themselves.

### BACKGROUND

It has been conclusively proven that a person’s mood may be determined with reasonable accuracy solely by tracking external physical parameters such as heartbeat rate and skin temperature. Many studies have been carried out in this field, but relatively few efforts have been made to commercialize this ability for consumer benefit.

At present there is no easily accessible device for detecting and tracking a user’s mood, let alone any product which can make such a determination and proactively use the acquired information to improve or stabilise a person’s mood.

In research the main method by which a person’s mood is measured is by electroencephalogram (EEG), a test that detects electrical activity in the brain using electrodes attached to the scalp to pick up communications between brain cells via electrical impulses. EEG tests and equipment are not accessible nor convenient for the general public, since they are expensive and require physical contact between a person’s head and multiple electrodes at all times.

WO2020104722A1 describes a device for determining the emotional state of a user, but the emphasis of the disclosure is for determining a likelihood that the user is at risk of attack, no protocols are disclosed for adjusting an environment of a user based on their mood.

US2012116186A1 describes a method and apparatus for remote evaluation of a subject’s emotive and/or physiological state. The disclosure is aimed at effective ways of conveniently measuring emotional state from a distance, and mostly avoids skin-contact methods. The only skin contact method disclosed is implemented on a smartphone which would not have continuous skin contact for prolonged periods of time.

EP2698685A2 describes a mobile handset device collects sensor data about the physiological state of the user of the handset. The purpose for the collection of the data is specifically intended for therapeutic purposes, and more specifically for informing a therapist with reliable data on the mood of a user. There is no disclosure of an algorithm for implementing mood-based protocols based on the determined data.

KR20190129532A describes an emotion determination system and a method which detect emotional responses of a user using multimedia content viewed on a smartphone and applies various stimuli to the user in response to the derived emotion type. The focus of the disclosure is on the emotional response of the user to multimedia content viewed on a screen, no environment changing protocols are initiated in response.

It is within this context that the present invention is set.

### SUMMARY

The present invention aims to provide a convenient and accessible method for any person to become more aware of their own emotions and reactions, and with an emphasis on using this new ability to initiate protocols for proactively maintaining a better mood and improving mental health and quality of life of a user. In particular, the invention takes advantage of measuring a user’s galvanic skin response as an important parameter for determining their mood, in combination with their skin temperature, comparing the measured values to a set of known thresholds to make a mood determination, and subsequently initiating one or more protocols to adapt an environment of a user to their mood. The methods and devices presented herein may also be used in conjunction with simulated environment technologies such as virtual and augmented reality devices.

Thus, according to a first aspect of the present invention, there is provided a method for determining an emotional state of a user, the method comprising: measuring, by a first sensor, a galvanic skin response voltage of a user; measuring, by a second sensor, a skin temperature of the user; receiving, by a processor, at least one measured value from the first sensor and at least one measured value from the second sensor; comparing, by the processor, the at least one measured value from the first sensor to a plurality of predefined threshold values for galvanic skin response voltage, each being associated with one or more likely emotional states; comparing, by the processor, the at least one measured value from the second sensor to a plurality of predefined threshold values for skin temperature, each being associated with one or more likely emotional states; and based on the comparisons of the first and second measured values, determining an emotional state of the user, and in response to the determination that the user is in a particular emotional state, initiating one or more protocols for adapting the physical or virtual environment of the user to that emotional state.

In some embodiments, the method further comprises: measuring, by a third sensor, a heartbeat rate of the user; receiving, by the processor, at least one measured value from the third sensor; and comparing, by the processor, the at least one measured value from the third sensor to a plurality of predefined threshold values for heartbeat rates, each being associated with one or more likely emotional states; wherein the determination of the likely emotional state of the user is further based on the comparison for the third measured value.

In some embodiments, one or more of the at least one first measured value, the at least one second measured value, and the at least one third measured value comprise a series of values measured over a predefined time period, and one or more of the comparisons carried out by the processor are based on an average of the measured values in the respective series.

In some embodiments, the results of the comparisons are weighted in the determination of the emotional state of the user and the comparison for skin temperature values is given the most significance, followed by galvanic skin response voltage, followed by heartbeat rate.

In some embodiments, the method further comprises, in response to the determination that the user is in a particular emotional state, notifying the user or a specified third party of the emotional state.

In some embodiments, the method further comprises, in response to the determination that the user is in a particular emotional state, notifying an external device via wireless or internal communication.

In some embodiments, the method further comprises, in response to the determination that the user is in a particular emotional state, initiating one or more protocols for adapting the environment of the user to that emotional state by communication with one or more smart devices.

In some embodiments, the method further comprises determining or modifying predefined threshold values for one or more of the comparison operations based on a user profile and personal history.

In some embodiments the method further comprises: storing the results of one or more of the comparisons and/or the determination of the user mood in a database; verifying with the user whether the mood determination result was accurate; and, if the mood determination was not accurate, adjusting one or more predefined threshold values based on the stored data.

In some embodiments, initiating the one or more protocols comprises communicating with a smart home environment to adjust one or more of a temperature, a lighting hue and intensity, an odour, and an audio track or playback settings in the home environment.

In other embodiments, initiating the one or more protocols comprises communicating with a processor of a virtual reality or augmented reality device to adjust one or more of a visual scene, audio track, tactile response, and odour generated by the virtual reality or augmented reality device.

In further embodiments, initiating the one or more protocols comprises causing an audio track associated with the determined emotional state to be played to the user.

The audio track may be procedurally generated based on the determined emotional state.

According to a second aspect of the present invention, there is provided a device for determining an emotional state of a user, the device comprising: a first sensor configured to measure and output a galvanic skin response voltage when in bodily contact with a user; a second sensor configured to measure and output a skin temperature when in bodily contact with a user; a power unit; a wireless communication unit; and a controller configured to carry out the method of any one of the above-described embodiments.

In some embodiments, the device further comprises a third sensor configured to measure and output a heartbeat rate when in bodily contact with the user, and the controller is further configured to receive one or more values from the third sensor and output the one or more values to the external device for comparison.

In some embodiments, the one or more values comprise a series of values measured over a predefined time period.

In some embodiments, the device is in the form of a wearable. In other embodiments, the device is in the form of one of: a phone cover, a trackpad, a mouse, a keyboard, a joystick, a remote control, and a set of stickers.

In some embodiments, the wireless communication device comprises a Bluetooth module. In other embodiments the device communicates internally and in others it communicates via USB.

In some embodiments, the device is integrated with a virtual reality or augmented reality system.

### BRIEF DESCRIPTION OF THE DRAWINGS

Various embodiments of the invention are disclosed in the following detailed description and accompanying drawings.

FIG. 1 illustrates a block diagram schematic of an example embodiment of the device of the present invention and its internal components;

FIG. 2 illustrates an example embodiment of the device of the present invention in position on a user and in wireless communication with a mobile device of the user.

FIG. 3 illustrates a second example embodiment of the device of the present invention in position on a user’s wrist and in wireless communication with a mobile device of the user.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The aim of the invention is to provide a method and device that allows determination of a user’s overall mood based on physical parameters which are tracked constantly via bodily sensors, and that subsequently allow for protocols to be initiated to adapt an environment of the user to the determined mood to comfort them and increase mental wellbeing, in other words an emotion detection and influence technology.

Based on current research, the primary emotions that can be detected via such tracking are happiness, sadness and anger/distress, as these emotions cause measurable physiological changes with identifiable signatures as will be explained below. However, it is envisaged that in the future, ever more nuanced emotions will be detectable with reliable accuracy, especially once personal user data and history are incorporated for customised emotional profiles. The present invention is also applicable to such fine-tuned approaches, and while exemplified with respect to the above mentioned primary emotional states, it is not limited thereto.

Thus, provided herein is a computer implemented method for measuring or detecting a user’s emotional state, and a device capable of performing such measurements and communicating with an external entity such as a network or external device to record the determined emotional state with the aim of taking proactive actions based on the determined emotional state. For example, the device may comprise a wearable device having built in sensors and a wireless communication module configured to communicate the measurements to a processor with more processing power and which has access to one or more environment-changing utility functions.

The device consists of at least two physiological sensors that detect Galvanic Skin Response and skin temperature when in contact with the skin of a user. The measured parameters are translated into digital numbers for a processor of the device, in most cases a microprocessor. The processor either sends the received measurements to an external device for processing or has its own processor and dedicated software for analysing them, and establishes which emotion a user is perceiving. The algorithm compares the measured data to pre-established values and thresholds that are known to indicate certain emotional states for a person as is explained below.

Galvanic skin response is a surprisingly effective parameter for indicating emotional state and to date has not been utilised in a similar way to that disclosed in the present application.

Electrodermal activity (EDA) is the property of the human body that causes continuous variation in the electrical characteristics of the skin. Historically, EDA has also been known as galvanic skin response (GSR), a term which is used throughout this application to refer to electrodermal activity. Other terms used to refer to the same phenomenon include: electrodermal response (EDR), psychogalvanic reflex (PGR), skin conductance response (SCR), sympathetic skin response (SSR) and skin conductance level (SCL).

Specifically, Galvanic skin response (GSR) is a term that refers to the voltage measured between two electrodes placed on the skin of a subject without any externally applied current. It is measured by connecting the electrodes to a voltage amplifier. The electrodes are normally placed about an inch apart, and the voltage recorded varies according to the emotional state of the subject.

GSR varies with the state of sweat glands, and sweating is controlled by the sympathetic nervous system. If the sympathetic branch of the autonomic nervous system is highly aroused, then sweat gland activity also increases, which in turn increases GSR. Furthermore, GSR is not under conscious control. Instead, it is modulated autonomously by sympathetic activity which drives human behavior, cognitive and emotional states on a subconscious level.

GSR is thus an indication of psychological or physiological activity, and can be used as a reliable measure or insight into autonomous emotional regulation.

The relationship between emotional arousal and sympathetic activity alone is not enough to accurately identify which specific emotion is being elicited. However, the autonomic sympathetic changes also alter sweat and blood flow, which in turn affects other physiological measurables such as skin temperature and heartbeat rate. In combination with these other measurables, which tend to be higher or lower for particular emotional states, it is possible to define a set of thresholds which indicate the broad emotional state of a person with reliable accuracy.

The response of the skin and muscle tissue to external and internal stimuli can cause the conductance between two electrodes to vary by several microsiemens. A correctly calibrated device can thus record and display the subtle changes.

The number of sweat glands varies across the human body, being highest in hand and foot regions, in the range of 200-600 sweat glands per cm². As such, a preferred embodiment of the present invention is a wearable which allows the electrodes of GSR sensor of the device to maintain continuous contact with the skin of a user’s hand or wrist.

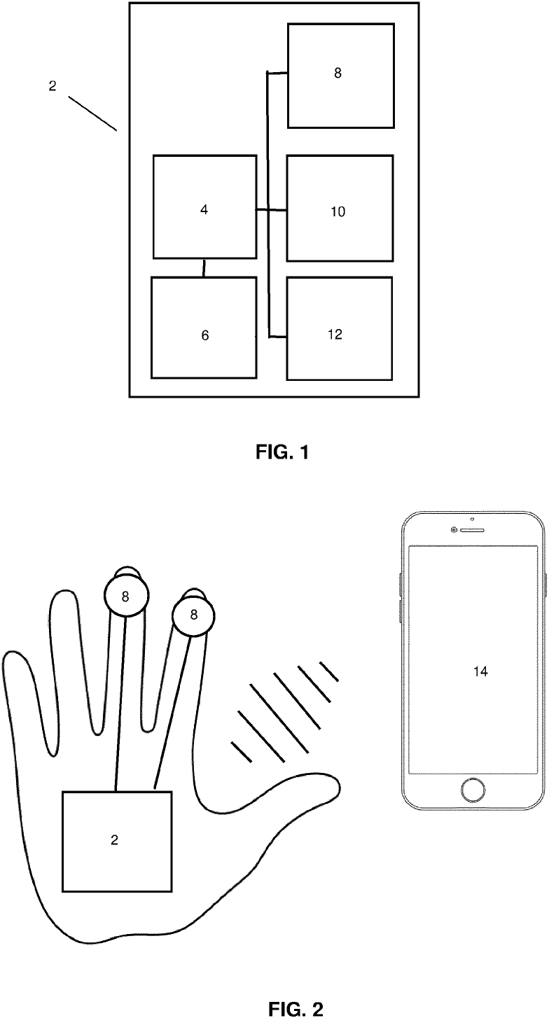

Thus, referring to FIG. 1, an example embodiment of the device 2 of the present invention is shown.

The device 2 comprises a controller in the form of a microprocessor 4, a wireless communication module 6 comprising a transceiver capable of sending and receiving signals from external devices and networks, and three physiological sensors: a Galvanic Skin Response sensor 8, a skin temperature sensor 10, and a heartbeat sensor 12. These sensors are attached to an electrical circuit board that translates the measured voltages into digital numbers and forwards them to the processor of the device. The device also comprises a power module for powering the aforementioned components which is not illustrated.

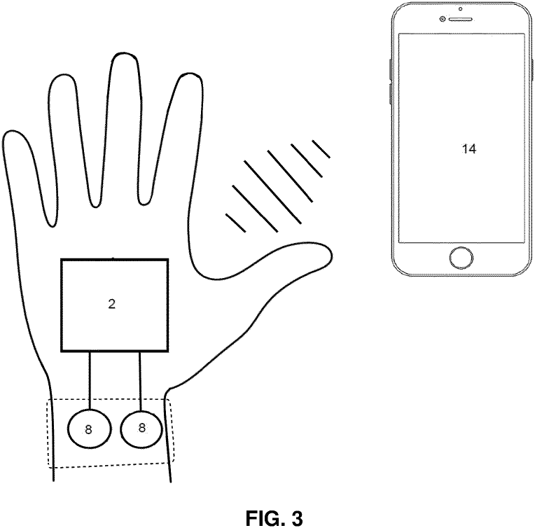

Referring to FIG. 2, a diagrammatic view of an example embodiment of the device 2 is shown.

Preferably, the GSR is measured by two separate electrodes placed on two different fingertips of the same hand of a user, thus in the example embodiment shown the device 2 takes the form of a wearable on the hand of the user and the GSR sensor comprises at least two electrodes 8 arranged to be placed in physical contact with different fingertips of the user, each of the electrodes being connected to the controller/microprocessor 4 of the device by wired connection.

Referring to FIG. 3, a diagrammatic view of a second example configuration of the device 2 is shown.

In this configuration the device is comprised in a wearable worn about a user’s wrist, that allows the two electrodes 8 to be in contact with separated skin surfaces on the wrist and to take their measurements from there.

In either case, the processor has installed thereon software that analyses the measured values and establishes through an algorithm what emotion the user is experiencing. This analysis may be carried out by the processor independently or in conjunction with a processor of an external device in wireless communication with the processor.

The algorithm compares the incoming data to pre-established thresholds, giving higher priority to skin temperature, then galvanic skin response, and ultimately the heartbeat of the user in beats per minute. Once the emotional state of the user has been established, one or more protocols for adapting an environment of the user to the emotional state are initiated.

Generally, the protocols involve communication with an external device having environment modifying capabilities. This may be as simple as causing a smartphone of a user to play an audio track associated with the determined mood, or may involve more significant modifications such as communicating with a smart home environment of the user to modify anything from lighting arrangements, temperature regulation, and even smell.

If audio is incorporated into the initiated protocol, the audio may in some cases be procedurally generated audio, with the algorithm generating the audio being selected based on the determined emotional state, and the algorithm may adjust the nature of the generated audio as changes in the user’s mood are detected.

Temperature has been shown to have an impactful effect on the emotional states of humans, as such temperature changes will often be incorporated into the initiated protocol where possible.

In one implementation, the method comprises altering the temperature by communication with a smart home environment in increments determined by the following equation:

$$\text{Δ} T = \frac{1}{1 + {e}^{\left(h ∗ \left(t - {t}_{0}\right)\right)}}$$

Where ΔT is the increment, t₀ is a set temperature programmed into the algorithm as an equilibrium temperature, and t the current known temperature which must be between 18 and 40° C. for the next increment to be added/subtracted to maintain the environment within a comfortable temperature range. An appropriate value for t₀ has been determined to be approximately 21° C., but other values may be used.

In some cases, the device 2 may be integrated or in communication with a virtual or augmented reality system the user is experiencing, and the protocols may involve introducing visuals associated with the determined emotional state. For example, a scene may be depicted to give the user a sense of warmth. This may be done in an obvious fashion or in a subtle fashion to subconsciously influence the mood of the user.

Other, more extreme visual representations of a user’s determined emotional state may also be incorporated, such as for example projecting red spikes coming out of walls when a user is angry, projecting blue waves surrounding a user when they are determined to be sad, etc. Such projections may also accompany other changes, such as projecting snowflakes falling about the user as the temperature is dropped, or orange lines surrounding the user when the temperature is raised.

Whether the lighting/visual change is done in a real environment by a smart home system or in a virtual or augmented environment, the underlying colour composition of the lighting is always important in influencing the emotional state of a user.

In some implementations, a warm white colour RGB composition of [245, 245, 210] is used in response to a detected calm emotional state of a user, a yellow colour RGB composition of [255, 255, 102] is used in response to a detected happy state of a user, a blue colour RGB composition of [0, 0, 255] is used in response to a detected sad emotional state of a user, and a red colour RGB composition of [255, 0, 0] is used in response to a detected angry emotional state of a user. Naturally varying shades of colour compositions will be used during transitions between the listed compositions.

In systems where it is possible, this visual change may be accompanied by haptic feedback such as vibrations at frequencies associated with the determined emotional state.

The chosen protocol to initiate in response to a determined emotional state may be for a specific medical, educational, entertainment, or research-based purpose. Furthermore, the associations between determined emotional states and the protocols to initiate in response to said states may be managed and constantly updated by a machine learning algorithm.

Furthermore, in systems where it is possible, even odour can be incorporated into the environmental change protocol. For example, a particular scent associated with a detected mood could be sprayed in the room using automated devices or directly released from the virtual reality headset in response to a detection of that mood.

The disclosed method may also be integrated with adaptive experiences such as adapted video games.

In the present example, to ensure no outlying measurements skew the resulting emotional determination, before the device outputs what emotion has been detected, the sensors will collect data for a predefined period such as for example over a period of 30 seconds, taking an average for each parameter. The data collected is used to tailor the parameters of emotional determination in a positive cycle, for example through verification by communication - using a wireless communication module of the device to check with the user whether previous determinations were correct. The wireless communication may for example be Bluetooth communication, however various implementations are possible as explained below.

In the illustrations of the example embodiments of FIG. 2 and FIG. 3 the device is shown in wireless communication with a mobile device 14 of a user.

The results of the emotional determinations will be continuously update and stored. Optionally the verifications can also be sent to a central database to continuously refine the predefined threshold settings.

In other embodiments not illustrated here, the device can take many other forms that are able to conveniently and non-intrusively come into contact with a user’s wrist or the fingertips of a user’s hand. For example, other types of wearable technology, phone covers, trackpads, keyboards, a computer mouse and joysticks. The device could even be a standalone device or a set of stickers containing sensors which could be placed anywhere the user desires.

Secondary code and functions that can be implemented once the user emotion has been detected such as alerting the user of their emotional state via pop-up notification via wireless communication with their mobile device.

One possible use of the method described herein is in medical situations where patients struggle to communicate their feelings or suffer distress and anxiety. The device could be used to communicate this quickly and easily to a medical professional responsible for their care. This application could be mirrored and expanded for other professionals and even for favourite contacts of the user. For example, notifications could be sent in all kinds of different situations to a therapist, a tutor, a friend of the user or even the police.

Furthermore, it is inevitable that widespread use of the device and method of the present disclosure will result in the accumulation of a large amount of data relating to the emotional states of various users in different situations, as well as the accuracy and fine-tuning of the measured parameter thresholds for determining user emotional states.

Thus, not only will the measured parameter thresholds constantly be improved and refined to improve the experience to the user, such as for example by accounting for slight differences between young and old people, male or female, or people that have different blood pressures, but additional applications of the newfound knowledge of external factors influencing the emotional states of the user could be developed.

For example, the introduction of a GPS feature to facilitate the tracking of an emotional map of places that make the user feel better or worst would be possible, as well as possible tracking and coordination of the interactions between maps of different users, allowing for determinations of whether there are people that cause a user to feel more relaxed or anxious. Such implementations would require further external and internal factors to be monitored either by the device itself or by an external device associated by the user which is in wireless communication with the device of the present invention. Activities would need to be tracked such as running and exercising, watching movies, playing games, attending gigs or amusement parks as all of these situations can affect the tracking of the parameters.

The user data collected and stored on remote servers can be utilised in a variety of ways. For example, in some implementations the data is used to optimise the emotional parameter thresholds in achieving an emotional state labelled the “status of calm”.

In one set of experiments carried out during the development of the disclosed device, data was collected before and during application of the device sensors to a set of subjects, tracking a visual graph of the three parameters through time. The emotional state determined by the device was compared to McNair’s Profile of Mood States (POMS) questionnaire, to verify the authenticity of the moods detected. The results were then analysed in order to narrow the ranges of each emotion, the analysis methods including: taking an average between the starting data collected for each emotion and the POMS to establish a new “truer” parameter around which to create the range for that specific emotion; data collection of emotional peaks happening during the experience which have been used to understand if the parameters have a wider or slighter range for each emotion; and determining the effects of playing contrasting music to the subject mood during measurement to verify if they would fall within the pre-defined ranges of moods that had been set.

Using such methods, the detection of emotions can be constantly improved via patches/software updates sent wirelessly from external devices and networks to the device controller, while the personal adaptation can be implemented in the basic code.

The operations described herein may be carried out by any processor. In particular, the operations may be carried out by, but are not limited to, one or more computing environments used to implement the method such as a data center, a cloud computing environment, a dedicated hosting environment, and/or one or more other computing environments in which one or more assets used by the method re implemented; one or more computing systems or computing entities used to implement the method; one or more virtual assets used to implement the method; one or more supervisory or control systems, such as hypervisors, or other monitoring and management systems, used to monitor and control assets and/or components; one or more communications channels for sending and receiving data used to implement the method; one or more access control systems for limiting access to various components, such as firewalls and gateways; one or more traffic and/or routing systems used to direct, control, and/or buffer, data traffic to components, such as routers and switches; one or more communications endpoint proxy systems used to buffer, process, and/or direct data traffic, such as load balancers or buffers; one or more secure communication protocols and/or endpoints used to encrypt/decrypt data, such as Secure Sockets Layer (SSL) protocols, used to implement the method; one or more databases used to store data; one or more internal or external services used to implement the method; one or more backend systems, such as backend servers or other hardware used to process data and implement the method; one or more software systems used to implement the method; and/or any other assets/components in which the method is deployed, implemented, accessed, and run, e.g., operated, as discussed herein, and/or as known in the art at the time of filing, and/or as developed after the time of filing.

As used herein, the terms “controller”, “microprocessor”, “computing system”, and “computing device”, include, but are not limited to, a virtual asset; a server computing system; a workstation; a desktop computing system; a mobile computing system, including, but not limited to, smart phones, portable devices, and/or devices worn or carried by a user; a database system or storage cluster; a switching system; a router; any hardware system; any communications system; any form of proxy system; a gateway system; a firewall system; a load balancing system; or any device, subsystem, or mechanism that includes components that can execute all, or part, of any one of the processes and/or operations as described herein.

As used herein, the terms computing system and computing entity, can denote, but are not limited to, systems made up of multiple: virtual assets; server computing systems; workstations; desktop computing systems; mobile computing systems; database systems or storage clusters; switching systems; routers; hardware systems; communications systems; proxy systems; gateway systems; firewall systems; load balancing systems; or any devices that can be used to perform the processes and/or operations as described herein.

Unless specifically stated otherwise, as would be apparent from the above discussion, it is appreciated that throughout the above description, discussions utilizing terms such as, but not limited to, “activating”, “accessing”, “adding”, “applying”, “analyzing”, “associating”, “calculating”, “capturing”, “classifying”, “comparing”, “creating”, “defining”, “detecting”, “determining”,, “eliminating”,, “extracting”,, “forwarding”, “generating”, “identifying”, “implementing”, “obtaining”, “processing”, “providing”, “receiving”, “sending”, “storing”, “transferring”, “transforming”, “transmitting”, “using”, etc., refer to the action and process of a computing system or similar electronic device that manipulates and operates on data represented as physical (electronic) quantities within the computing system memories, resisters, caches or other information storage, transmission or display devices.

Those of skill in the art will readily recognize that the algorithms and operations presented herein are not inherently related to any particular computing system, computer architecture, computer or industry standard, or any other specific apparatus. Various general purpose systems may also be used with programs in accordance with the teaching herein, or it may prove more convenient/efficient to construct more specialized apparatuses to perform the required operations described herein. The required structure for a variety of these systems will be apparent to those of skill in the art, along with equivalent variations. In addition, the present invention is not described with reference to any particular programming language and it is appreciated that a variety of programming languages may be used to implement the teachings of the present invention as described herein, and any references to a specific language or languages are provided for illustrative purposes only and for enablement of the contemplated best mode of the invention at the time of filing.

The present invention is well suited to a wide variety of computer network systems operating over numerous topologies. Within this field, the configuration and management of large networks comprise storage devices and computers that are communicatively coupled to similar or dissimilar computers and storage devices over a private network, a LAN, a WAN, a private network, or a public network, such as the Internet.

It should also be noted that the language used in the specification has been principally selected for readability, clarity and instructional purposes, and may not have been selected to delineate or circumscribe the inventive subject matter. Accordingly, the disclosure of the present invention is intended to be illustrative, but not limiting, of the scope of the invention, which is set forth in the claims below. In addition, the operations shown in the figures, or as discussed herein, are identified using a particular nomenclature for ease of description and understanding, but other nomenclature is often used in the art to identify equivalent operations.

What is claimed is:

1. A method for determining an emotional state of a user, the method comprising: measuring, by a first sensor, a galvanic skin response voltage of a user; measuring, by a second sensor, a skin temperature of the user; receiving, by a processor, at least one measured value from the first sensor and at least one measured value from the second sensor; comparing, by the processor, the at least one measured value from the first sensor to a plurality of predefined threshold values for galvanic skin response voltage, each being associated with one or more likely emotional states; comparing, by the processor, the at least one measured value from the second sensor to a plurality of predefined threshold values for skin temperature, each being associated with one or more likely emotional states; based on the comparisons of the first and second measured values, determining a likely emotional state of the user; and in response to the determination that the user is in a particular emotional state, initiating one or more protocols for adapting the environment of the user to that emotional state.

2. A method according to claim 1, wherein the method further comprises: measuring, by a third sensor, a heartbeat rate of the user; receiving, by the processor, at least one measured value from the third sensor; and comparing, by the processor, the at least one measured value from the third sensor to a plurality of predefined threshold values for heartbeat rates, each being associated with one or more likely emotional states; wherein the determination of the likely emotional state of the user is further based on the comparison for the third measured value.

3. A method according to claim 1, wherein one or more of the at least one first measured value, the at least one second measured value, and the at least one third measured value comprise a series of values measured over a predefined time period, and wherein one or more of the comparisons carried out by the processor are based on an average of the measured values in the respective series.

4. A method according to claim 1, wherein the results of the comparisons are weighted in the determination of the emotional state of the user and the comparison for skin temperature values is given the most significance, followed by galvanic skin response voltage, followed by heartbeat rate.

5. A method according to claim 1, wherein the method further comprises, in response to the determination that the user is in a particular emotional state, notifying the user of the emotional state.

6. A method according to any claim 5, wherein notifying the user comprises sending a notification to an external device via wireless communication or using pop up notifications via internal communication.

7. A method according to claim 1, the method further comprises determining or modifying the predefined threshold values for one or more comparison operations based on a user profile and personal history.

8. A method according to claim 1, wherein the method further comprises storing the results of one or more of the comparisons and/or the determination of the user mood in a database; verifying with the user whether the mood determination result was accurate; and if the mood determination was not accurate, adjusting one or more predefined threshold values based on the stored data.

9. A method according to claim 1, wherein the method further comprises storing the results of each comparison and determination of the user mood in a database as well as tracking the location of the user by GPS, thereby enabling the moods of the user to be correlated with specific locations and events.

10. A method according to claim 1, wherein initiating the one or more protocols comprises communicating with a smart home environment to adjust one or more of a temperature, a lighting hue and intensity, an odour, and an audio track in the home environment.

11. A method according to claim 1, wherein initiating the one or more protocols comprises communicating with a processor of a virtual reality or augmented reality device to adjust one or more of a visual scene, audio track, tactile response, and odour generated by the virtual reality or augmented reality device.

12. A method according to claim 1, wherein initiating the one or more protocols comprises causing an audio track associated with the determined emotional state to be played to the user.

13. A method according to claim 12, wherein the audio track is procedurally generated based on the determined emotional state.

14. A device for determining an emotional state of a user, the device comprising: a first sensor configured to measure and output a galvanic skin response voltage when in bodily contact with a user; a second sensor configured to measure and output a skin temperature when in bodily contact with a user; a power unit; a wireless communication unit; and a controller configured to receive one or more values from each of the first and second sensors and to carry out the method of claim 1 either independently or in conjunction with an external device in wireless communication with the controller via the wireless communication unit.

15. The device of claim 14, wherein the device is in the form of a wearable.

16. The device of claim 14, wherein the device is in the form of one of: a phone cover, a trackpad, a mouse, a keyboard, a joystick, and a s et of stickers.

17. The device of claim 14, wherein the wireless communication device comprises a Bluetooth module.

18. The device of claim 14, wherein the device further comprises a location tracking module having GPS capabilities, and wherein the controller is further configured to store the location of the device each time a mood of a user is determined, thereby enabling correlations to be determined between specific locations or events and a user mood.

19. The device of any of claim 14, wherein the device is integrated with a virtual reality or augmented reality system.
